(19) United States
(12) Patent Application Publication
(10) Pub. No.: US 2007/0209957 A1
(43) Pub. Date: Sep. 13, 2007

(54) Packaging system for medical devices

(75) Inventors: Scott Glenn, Cordova, TN (US); Robin L. Turner, Memphis, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/370,896

(22) Filed: Mar. 9, 2006

(51) Int. Cl. B65D 73/00; A61B 19/02

(52) U.S. Cl. 206/462; 206/438

(57) ABSTRACT

Packaging systems for medical devices, such as surgical instruments, implants, and prostheses, are provided herein. In one embodiment, the packaging system comprises a blister tray that is at least semi-transparent and has at least one cavity capable of containing a medical device or portion thereof. The system additionally comprises a card having at least one window, wherein the at least one cavity in the blister tray protrudes through the at least one window in the card. The packaging systems provide a convenient, informative, and preferably sterile means of distributing medical devices.

### FIELD OF THE INVENTION

Embodiments relate to packaging systems for medical devices such as surgical instruments, implants, prostheses, and the like. More specifically, the embodiments relate to blister packaging systems for the distribution, storage, and marketing of medical devices.

### BACKGROUND

Modern medicine increasingly utilizes medical devices such as surgical instruments, implants, prostheses, and the like. Implants and prostheses are medical devices that typically augment or replace body structures that are deteriorated, damaged, injured, or otherwise compromised. Exemplary medical implants and prostheses include cardiovascular implants, arterial stents, heart valves, orthopedic implants, dental implants, bone screws, plates, drills, bits, and so forth. Surgical instruments are medical devices that assist a surgeon in performing an operation. Exemplary surgical instruments include forceps, drivers, scapels, cathetars, trocars, syringes, drill bits, endoscopes, and so forth.

Medical devices typically must be sterilized prior to use in order to prevent infection of the patient. Generally, sterilization of the medical device can be done anytime before, during, or subsequent to packaging of the device, so long as the device is sterilized sometime before use. Thus, the medical device typically may be sterilized and ready for use immediately upon removing the device from its packaging. This assumes, of course, that the medical device's packaging is capable of maintaining the device's sterility subsequent to undergoing a sterilization procedure, whether that procedure precedes, occurs during, or is subsequent to packaging of the device.

Choosing an appropriate medical device for use in a given medical procedure may be difficult, if not impossible, to accomplish prior to surgery. Therefore, selection of a medical device, especially a medical implant or prosthesis, for use in medical procedure sometimes must occur during the course of surgery. If the medical device is packaged in a non-transparent packaging material, then it may be necessary to break open the packaging in order to examine the device and determine if it is appropriate for use, thereby exposing the device to a non-sterile external environment. Therefore, opening the packaging material of the medical device in order to inspect the device and determine its utility for a given medical procedure may prevent the subsequent use of the device until it has been re-sterilized and re-packaged.

Besides maintaining a medical device's sterility, another purpose of medical packaging is to protect the packaged device from tampering, and to maintain its authenticity. For example, medical packaging typically is sealed in order to prevent access to the device packaged inside. Once the seal has been broken, it is no longer possible to guarantee that the medical device in the packaging is authentic and conforms to the product standards of the manufacturer. Thus, opening the packaging material of the medical device in order to examine the device and determine if it is appropriate for use also may prevent the subsequent use of the device until it has been inspected, re-sealed, and re-packaged by an authorized party. Those skilled in the art will appreciate that inspection, re-sealing, re-packaging, and sterilization impose significant costs when the wrong device is opened.

### BRIEF SUMMARY

What is needed are medical packaging system that are capable of maintaining medical devices in a sterile environment. Additionally, what is needed are packaging systems that allow a medical device to be visually inspected and examined without opening the packaging.

Therefore, in accordance with an embodiment, there is provided a packaging system for medical devices. The system comprises a blister tray that may be at least semi-transparent and has at least one cavity. The cavity may be capable of containing a medical device or a portion thereof. The system further comprises a card having at least one window. The at least one cavity in the blister tray may protrude through the at least one window in the card.

In accordance with another embodiment, there is provided a packaging system for medical devices comprising a blister tray that is at least semi-transparent and has at least one cavity. The system further comprises a blister tray closure removably disposed over the blister tray and covering the cavity in the blister tray. Additionally, the system comprises a carton having at least one window. The blister tray may be positioned inside of the carton and the at least one cavity in the blister tray may be substantially visible through the at least one window in the carton.

These and other features and advantages of the embodiments will be apparent from the description provide herein.

### BRIEF DESCRIPTION OF THE DRAWINGS

FIG. 1 illustrates an exemplary carton-type packaging system from the prior art.

FIG. 2, embodiments A and B, illustrates an exemplary card-type packaging system according to the embodiments.

FIG. 3 illustrates an exemplary blister tray and associated card with a window and backing card.

FIG. 4 illustrates an exemplary blister tray and clam-shell card arrangement.

FIG. 5 illustrates an exemplary clam-shell packaging system.

FIG. 6 illustrates an exemplary card-type packaging system comprising two blister trays.

FIG. 7 illustrates an exemplary carton-type packaging system according to the embodiments.

FIG. 8 illustrates an exemplary carton-type packaging system and a medical device contained therein.

FIG. 9 illustrates an exemplary card-type packaging system having measuring indicia on its exterior.

### DETAILED DESCRIPTION OF THE PREFERRED EMBODIMENTS

The following description is intended to convey a thorough understanding of the various embodiments by providing a number of specific embodiments and details involving packaging systems for the distribution, storage, and marketing of medical devices. It is understood, however, that the embodiments are not limited to these specific embodiments and details, which are exemplary only. It is further understood that one possessing ordinary skill in the art, in light of known systems and methods, would appreciate the use of the embodiments for their intended purposes and benefits in any number of alternative embodiments.

As used herein and in the appended claims, the singular forms “a,” “an,” and “the” include plural reference unless the context clearly dictates otherwise. Thus, for example, a reference to “a window” includes a plurality of windows, and a reference to “a device” is a reference to one or more devices, and so forth.

The expression “medical device,” as used herein, denotes various surgical instruments, prostheses, and implantable devices used in the field of medicine. In general, the packaging systems described in the embodiments may be used to package one or more medical devices, or portions thereof, in a single packaging system. The packaging systems described in the embodiments therefore may be widely applicable in the distribution, storage, and marketing of various medical devices, such as surgical instruments, prostheses, and implantable devices.

Surgical instruments include, but are not limited to, burrs, cannulas, catheters, cauterizers (including tips and handles), chisels (e.g., bone chisels), clamps (e.g., anastomosis, bulldog, kidney, and kidney pedicle clamps), curettes (e.g., bone curettes), distractors, drill bits, electrodes, endoscopes, evacuators, files (e.g., bone files and rasps), forceps (e.g., arthroscopy, biopsy, grasping, hemorrhoidal, hook-punch, intestinal, and stomach forceps), hooks (e.g., exploring, sharp hooks, and sinus renalis hooks), irrigating tubes, knives (e.g., arthroscopy and meniscus knives), laproscopy devices, needles (e.g., biopsy, guide, ligature, micro, puncture, reverdin, suture, and verres needles), obturators, probes (e.g., duct and fistual probes), reamers (e.g., bone and medullary reamers), retractors (e.g., abdonminal, balfour, deaver, delicate, double-ended, dura, knee, Kocher, Langenbeck, malleable, pelvis, rectal, self-retaining, Volkmann, and Weitlaner retractors), ring cutters, rongeurs (e.g., bone rongeurs), saws (e.g., amputation and metacarpal saws), scalpel blades and handles, scissors (e.g., delicate, dissecting, hook, Mayo, Metzenbaum, micro, and operating scissors), scoops, scopes, (e.g., anosopes, arthroscopes, endoscopes, proctoscopes, and sphincteroscopes) screw drivers, stapels, suture apparati (e.g., clips, hooks, and wire), syringes, trocars, tubes (e.g., aspirating and suction tubes), vein strippers, and so forth. One of skill in the art will appreciate many other surgical instruments that may be packaged using the packaging systems provided by the embodiments.

Prostheses and implantable devices include, but are not limited to, articular cups (e.g., acetabular cups), bone pins, bone screws, cosmetic implants (e.g., breast implants), defibrillators, dental prostheses, external fixation devices (especially pins connecting external fixation devices to internal bony structures), facet arthroplasty devices, femoral head implants, femoral stem implants, heart valves, hip screws, hip sockets, humeral head implants, humeral stem implants, internal fixation rods, intervertebral disc devices (such as total disc replacements, nucleus replacements, and fusion cages), intramedullary nails, knee minicus implants, nail and screw orthopedic plates (e.g., calcaneal, distal radius, fibula, meta, mini condylar, spider, straight, tubular, L-, and T-plates), pacemakers, penile implants, pins (e.g., clavicle and hip pins), spinal fixation systems (e.g., fixation rods, screws, nails, etc.), stents (e.g., arterial and urinary track stents), total ankle replacement devices, total knee replacement devices, and so forth. One of skill in the art will appreciate many other prostheses and implantable devices that may be packaged using the packaging systems provided by the embodiments.

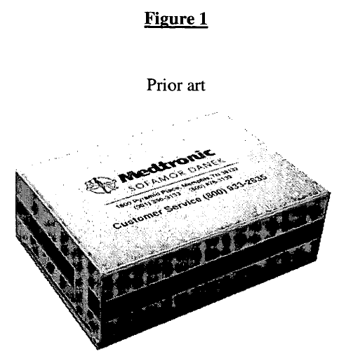

FIG. 1 illustrates an exemplary carton-type packaging system from the prior art. In FIG. 1, the prior art packaging system does not allow visual inspection of the medical device packaged therein from outside of the package. Therefore, in order to inspect the packaged medical device, a surgeon may need to break the seal on the package and remove the device contained therein. Removing the medical device may contaminate it if it previously had been sterilized. Furthermore, opening the package may void the manufacturer's warranty of authenticity and could potentially subject the medical device to tampering. Thus, if the medical device is determined to be inappropriate for immediate use, it may need to be returned to the manufacturer or another authorized party for processing (e.g., sterilization) and re-packaging. In some instances, medical device manufacturers have required that devices in opened packages be purchased by the party that opens the package, regardless of whether or not the packaged device is eventually used in surgery. Thus, prior art packaging systems may create extra costs and inconvenience that are borne by medical care providers, insurance carriers, and patients.

Accordingly, the embodiments herein provide packaging systems for medical devices that may overcome some or all of the disadvantages of the prior art packaging systems. For example, the packaging systems described in the embodiments may allow visual inspection of the medical devices from outside of the package, without requiring that the packaging be opened. Furthermore, the packaging systems of the embodiments may be capable of maintaining a medical device packaged therein in a sterile environment while simultaneously allowing visual inspection of the device. Also, the packaging systems of the embodiments may present additional information to a surgeon or other medical practitioner regarding the medical devices packaged therein. These features of the packaging systems described in the embodiments may assist a surgeon in determining the suitability of the medical device for use in a given medical procedure or surgery, without requiring that the device be removed from its packaging.

In one embodiment, there is provided a packaging system for medical devices. The system comprises a blister tray that is at least semi-transparent and has at least one cavity. The cavity is capable of containing a medical device or a portion thereof. The system further comprises a card having at least one window. The at least one cavity in the blister tray protrudes through the at least one window in the card.

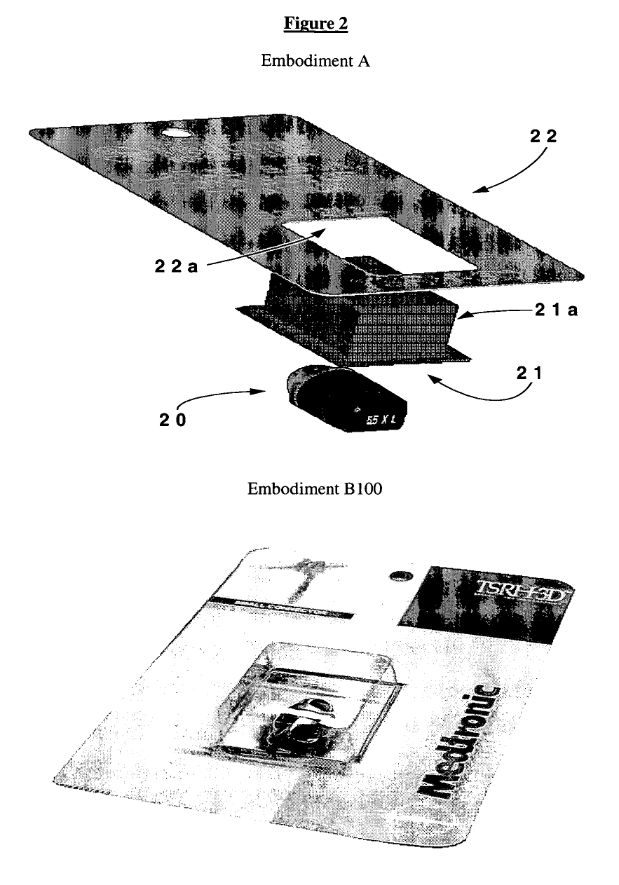

FIG. 2, embodiment A, illustrates an exemplary card-type packaging system according to the embodiments. A medical device 20 is provided, in this case a connector for use with a rod-style spinal fixation system. The device fits into a cavity 21a in a blister tray 21. A card 22 having a window 22a also is provided, through which the cavity 21a in the blister tray 21 is capable of protruding. FIG. 2, embodiment B, illustrates the card-type packaging system in an assembled condition. The blister tray preferably is made of a transparent or semi-transparent polymer material such that the medical device positioned in the cavity of the blister tray may be capable of being viewed through the blister tray. The blister tray also may be made from opaque materials. Exemplary polymeric materials from which the blister tray may be molded include, but are not limited to, polyethylene teraphthalate (“PET” or “PETE”), polyethylene teraphtalate glycol (“PETG”), high density polyethylene (HDPE), low density polyethylene (LDPE), polyvinyl chloride (PVC), polypropylene (PP), polystyrene (PS), high impact polystyrene (HIPS), polycarbonate (PC), acrylonitrile butadiene styrene (ABS), and other appropriate polymeric materials, as will be appreciated by one of skill in the art.

The card having at least one window preferably is an opaque, typically paper-based material. The card may provide additional surface area to the packaging system, on which information about the medical device's manufacturer, the medical device, pricing, use of the medical device, warnings, and so forth may be printed. Furthermore, the additional surface area may enable the packaging system to be easily grasped, held, and handled, especially in the case of a small medical device, where the blister tray that holds the device is correspondingly small.

In alternative embodiments, one or more cavities may be provided in the blister tray. The one or more cavities may collectively or individually contain one or more medical devices and portions thereof. For example, a single blister tray may have three cavities, each of which contains a medical device (e.g., three bone screws of differing lengths, or three bone screws of the same length, or a driver and two bone screws, and so forth). In this way, a single package may contain multiple, preferably complementary, medical devices.

It is desirable to seal the blister tray in order to hold the medical device in place in the at least one cavity of the blister tray. Sealing of the blister tray may be accomplished via several different methods, including securing a blister cover to the blister tray and securing a backing card to the blister tray. The blister cover or backing card may close the cavity in the blister tray, thereby trapping and holding the medical device in the cavity. Preferably, the blister cover or backing card may be hermetically sealed to the blister tray in order to prevent the passage of infectious agents to the medical device that is sealed in the cavity in the tray. Sealing may be accomplished by the application of a suitable adhesive such as the CR-27® heat-sealing adhesive commercially available from Perfecseal, Oshkosh, Wis., to seal the blister cover or backing card to the blister tray. If desired, for extra protection, the blister tray may be sealed both with both a blister cover and a backing card.

Also, the blister cover or backing card preferably may be removable from the blister tray, so that the cover or card may be removed by a surgeon or other medical practitioner in order to access the medical device in the blister tray. This may be preferred to non-removable blister covers and backing cards because cutting or breaking a blister tray itself often is difficult, potentially time consuming, and may cause damage to the device when compared to simply removing the cover or card. Thus, the adhesive used to secure the blister cover or backing card to the tray may be chosen such that the resulting seal is not too difficult to break, when intended.

Blister covers may be flexible, peelable sheets of materials that are capable of being secured to the blister tray. Blister covers may be made from a wide variety of materials, including spun-bonded polymeric fabrics, such as the spun-bonded polyolefin fabric sold commercially under the name Tyvek® by Dupont, Wilmington, Del. Another exemplary blister cover is a metallic, in particular aluminum, foil-type material. For example, the blister cover may be an aluminum foil that is layered on either side with a polyolefin such as polyethylene.

Preferably, the medical device and at least the interior of the cavity in the blister tray may be sterilized either prior or subsequent to sealing the device in the cavity of the blister tray. In order to sterilize the medical device and interior of the cavity after sealing the device therein, at least one of the blister cover, blister card, or blister tray preferably may be permeable to a sterilizing agent such as gamma radiation or gaseous ethylene oxide. Thus, the material used for the blister cover, blister card, and blister tray may be limited in some instances to those materials that are permeable to the sterilizing agent that is desired to be used, if any. Other appropriate sterilizing agents include electron-beam (E-beam) radiation, gas plasma, ultraviolet radiation (UV), and hydrogen peroxide (H₂O₂).

In one embodiment, the blister tray may be positioned between the card having at least one window and a backing card. The two cards may be secured together, for example, using an adhesive. Additionally, the blister tray preferably may itself be secured to the backing card with, for example, an adhesive. The blister tray may be secured to the backing card by applying an appropriate adhesive to a lip or flange on the perimeter of the blister tray and pressing the adhesive-covered lip or flange of the blister tray to the backing card. Preferably, securing the blister tray to the backing card creates a hermetic seal around the cavity in the blister tray. In this way, the cavity in the blister tray and the medical device, at least a portion of which is positioned in the cavity, may be sterilized, if desired, either before or after sealing the blister tray to the backing card, and the sterilized environment maintained subsequent to sealing the tray to the card.

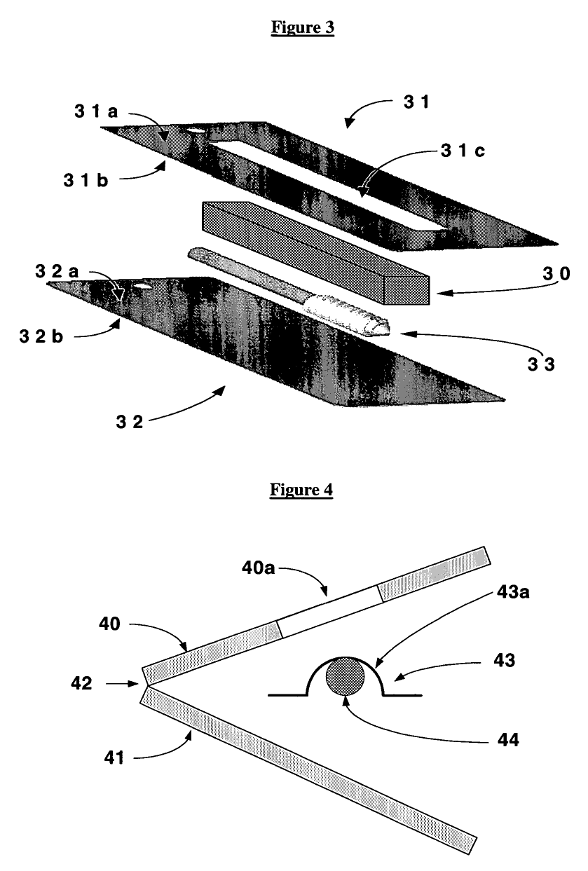

FIG. 3 illustrates an exemplary blister tray 30 ready to be positioned between a card 31 with at least one window 31c and a backing card 32. Card 31 has top 31a and bottom 31b surfaces. Similarly, backing card 32 has top 32a and bottom 32b surfaces. The two cards 31 and 32 may be secured together, for example, by placing an adhesive on the perimeter of the back 31b of card 31 and/or on the perimeter of the front 32a of the backing card 32. By securing cards 31 and 32 together, the blister tray 30 may be secured therebetween, with at least a portion of a medical device 33 in the cavity in the blister tray. Optionally, and especially if a sterile environment is desired, the blister tray 30 may be directly secured to the backing card 32 using an adhesive placed, for example, on the bottom of a flange or lip on the perimeter of the blister tray or on the corresponding portion of the top surface 32a of the backing card 32 to which the flange or lip will mate. In this way, the volume within the cavity of the blister tray can be sealed from the environment, and maintained sterile. It will be appreciated that, in an alternative embodiment, the blister tray may be sealed with a blister cover, and optionally additionally secured to the backing card using an adhesive, thus providing a package that may be capable of maintaining a sterile environment in the blister tray.

While this embodiment is illustrated with reference to a blister tray with a single cavity, it will be appreciated that more than one cavity may be present in the blister tray, and that the cavities each may hold one or more medical devices or portions thereof. For example, a two-cavity blister tray may hold several bone screws in one cavity, and a driver for the screws in the other cavity. Endless variations in multiple-cavity arrangements are possible. Additionally, it will be appreciated that the at least one window in the card may be large enough for more than one cavity to protrude therethrough. Thus, the packaging systems of the embodiments may comprise multiple cavities in the blister tray protruding through multiple windows in the card, each cavity holding one or more medical devices or portions thereof.

In another embodiment, a clam-shell card arrangement may be used to position the blister tray between opposing sides of a folded card. In this embodiment, a card having at least one window is folded into two sections. The two sections need not be equal in size, but preferably are at least approximately equal. The card preferably is folded so that the one or more windows in the card each are disposed entirely on either of the two sections of the card (i.e., so that the fold does not run through any of the windows). The blister tray may be placed against the card such that the at least one cavity in the blister tray protrudes through at least one window in the card. The two sections of the card may be folded together so that the blister tray is positioned between the two sections.

FIG. 4 illustrates an exemplary blister tray ready to be positioned between two section of a clam-shell card arrangement. A blister tray 43 having a cavity 43a is provided, as is a card. The card is folded at crease 42 into two sections 40 and 41. Section 40 has at least one window 40a therein. The cavity 43a in the blister tray is capable of protruding through the window 40a in the card. A medical device 44 or portion thereof is positioned in the cavity 43a in the blister tray 43. It will be appreciated that when the two sections of the card are folded towards each other, the blister tray will be positioned in-between the two sections and may be secured there. As in other embodiments, the two sections of the card may be secured together, for example, using a bead of adhesive around the perimeters of the faces of the two sections of the card that will mate together. Also, the blister tray may be secured to one or both sections of the card, for example, using a bead of adhesive around the perimeter, preferably on a flange or lip, of the blister tray. Again, the blister tray or a portion of the card may be comprised of a thermoplastic material (or other similar material) that can form a seal upon application of external energy. It will be appreciated that, in an alternative embodiment, the blister tray may be sealed with a blister cover, and optionally additionally secured to the clam-shell card, thus affording extra protection to the medical device contained in the blister tray.

In another alternative embodiment, the clam-shell arrangement may have two or more windows, and the windows may be disposed on one or more sections of the folded card. One or more cavities in the blister tray may protrude through the one or more windows of the card. In another embodiment, the clam-shell arrangement may have a card that is folded into more than two sections, wherein at least one of the sections comprises at least one window through which the cavity in the blister tray may protrude and at least one of the sections acts as a backing card, substantially covering the cavity in the blister tray. As in the other embodiments, it will be appreciated that, alternatively, the blister tray may be sealed with a blister cover, and optionally additionally secured to the card, thus affording extra protection to the medical device contained in the blister tray.

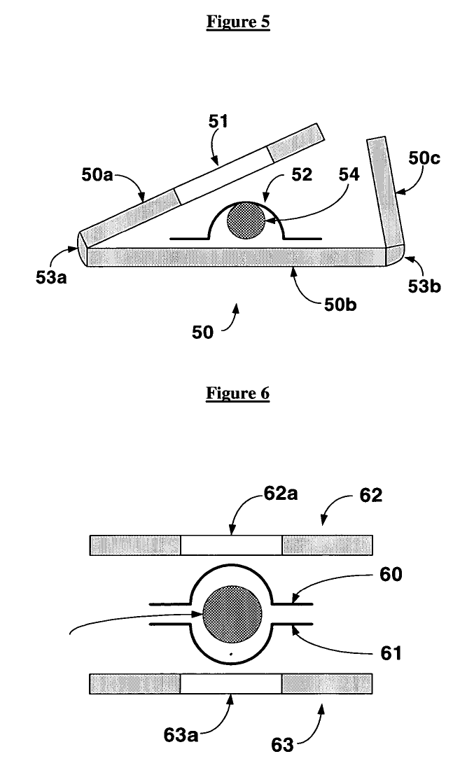

FIG. 5 illustrates an exemplary clam-shell packaging system of additional alternative embodiments. In FIG. 5, the card 50 is folded at 53a and 53b, dividing the card into three sections: 50a, which contains a window (51); 50b, which acts as a backing card to seal the cavity in the blister tray (optionally in combination with a blister cover); and 50c. The blister tray 52 is capable of holding a medical device 54 or a portion thereof. As in other embodiments, the blister tray 52 may be secured to the section of the card acting as a backing card 50b using an adhesive, for example, placed on a flange or lip around the tray's perimeter. Additionally, adhesive may be applied to one or more surfaces of card sections 50a, 50b, and 50c to secure the sections to one another and/or to the blister tray. The card may be folded such that section 50c is on top, 50a underneath, and 50b on the bottom. Or, the card may be folded so that section 50a is on top, 50c underneath, and 50b on the bottom.

In another alternative embodiment, two blister trays may be secured together with an adhesive such that the cavities in the two trays are aligned. The aligned cavities may form one or more volumes in which one or more medical devices or portions thereof may be positioned. The joined blister trays may be secured to a first card having at least one window, through which at least one of the cavities of the joined blister trays is capable of protruding. The joined blister trays also may be secured to a second card having at least one window, where the second card is placed on the side of the joined blister trays opposite the first card. This embodiment therefore provides windows on either side of the packaging system, so that the medical device may be viewed from either side of the package.

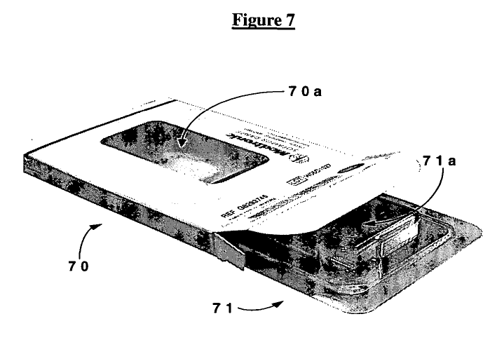

FIG. 6 illustrates an exemplary two-blister tray packaging system according to the embodiments. A first blister tray 60 and a second blister tray 61 are provided. The blister trays each may have one or more cavities, that, when they are aligned opposite to each other so that opposing surfaces of the trays are in contact, form one or more volumes. In each of the one or more volumes, one or more medical devices 64 or portions thereof may be contained. A first card 62 having at least one window 62a also is provided, through which the cavity in the first blister tray 60 is capable of protruding. A second card 63 having at least one window 63a additionally is provided, through which the cavity in the second blister tray 63 is capable of protruding. Skilled artisans will recognize that the two cards, although shown separately in FIG. 6, may be joined in one or more places in a clam-shell arrangement, or other similar arrangements. The blister trays may be joined together, for example, using an adhesive placed on the perimeter of the opposing surfaces of the trays, preferably on a flange or lip of the perimeter of the opposing surfaces of the trays. The blister trays may be secured to the cards using additional adhesive, or may simply be held in place by securing the cards to one another so that the blister trays are slightly compressed and snugly held in place between the cards. The cards themselves may be secured together, again, using an adhesive, preferably placed on the perimeters of their opposing surfaces. The medical device 64, at least a portion of which is contained in the volume comprising the cavities of the two blister trays 60 and 61, is capable of being viewed from either side of the packaging system.

In accordance with another embodiment, there is provided a packaging system for medical devices comprising a blister tray that is at least semi-transparent and has at least one cavity. The system further comprises a blister tray closure removably disposed over the blister tray and covering the cavity in the blister tray. Additionally, the system comprises a carton having at least one window. The blister tray is positioned inside of the carton and the at least one cavity in the blister tray is substantially visible through the at least one window in the carton.

The blister tray has been described previously. Generally, the blister tray may comprise a transparent or semi-transparent polymeric material, such as PET or PETG. The blister tray closure may be any appropriate closure for affixing to the blister tray, and more preferably closures that are capable of maintaining a sterile environment in the blister tray once it has been sterilized. The blister tray closure may be a blister cover or a backing card, both of which have been described previously. The blister cover, for example, may be a peelable polymeric or foil material that is secured to the back of the blister tray using an adhesive, or using the material itself and applying external energy. A backing card may be a paper-based material that also adheres to the blister tray by use of an appropriate adhesive. The blister tray closure thus seals the one or more cavities in the blister tray. Each of the one or more cavities is capable of containing one or more medical devices, or portions thereof.

Preferably, an appropriate adhesive is chosen when securing the blister tray closure to the blister tray in order to hermetically seal the blister cavity or cavities in the tray, so that the medical device or devices that are at least partially contained in the cavities can be maintained in a sterile environment. The medical device or devices and interior of the blister cavity or cavities may be sterilized before, during, or after assembly of the blister tray, for example, using a sterilizing agent such as gamma radiation or gaseous ethylene oxide.

The assembled blister tray, medical devices, and blister tray closure may be inserted into a carton having at least one window. The carton, like the card having at least one window and backing card previously described, may be a paper-based material. The one or more windows in the carton may be positioned so that the medical devices in the blister tray are substantially viewable through the windows in the carton. In this way, analogous to the blister tray/card combination, the medical device in the blister tray may be capable of being viewed and inspected from outside of the packaging, and without opening the packaging. Thus, the blister tray/carton combination may provide a packaging system that facilitates visual inspection of the medical devices packaged therein, without the deleterious effects of opening the packaging (e.g., contamination of the devices, voiding the manufacturer's warranty of authenticity, and so forth).

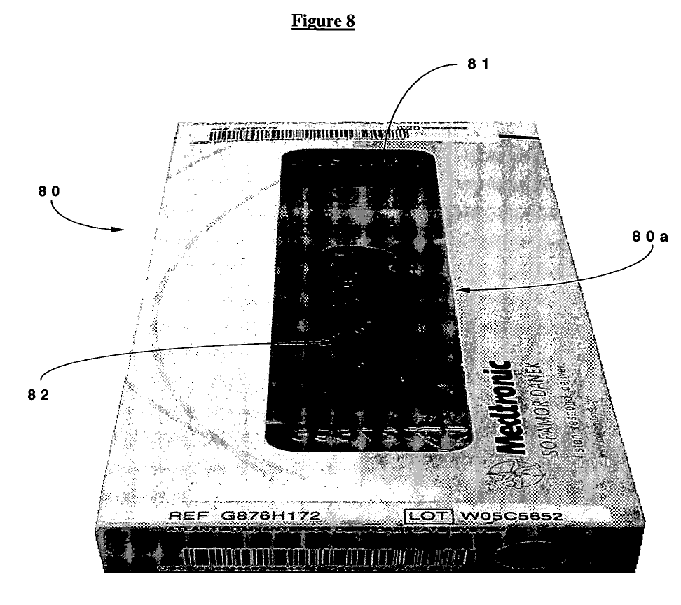

FIG. 7 illustrates a carton/blister tray combination in accordance with the embodiments. A carton 70 has a window 70a. A blister tray 71 having a cavity 71a may be placed in the carton 70. The cavity 71a is capable of holding one or more medical devices, or portions thereof, and is covered by an appropriate blister tray closure. The blister tray closure may be, for example, a blister cover (e.g., Tyvek® or an aluminum foil) or a backing card (i.e., a paper-based card) to which the blister tray is secured. While the carton has been illustrated with a single window, it should be appreciated that the carton may have one or more windows, placed in one or more of the carton's sides. Additionally, one or more cavities in the blister tray may be visible through each window. Furthermore, each of the cavities in the blister tray may hold one or more, or parts of one or more, medical devices. Therefore, one of skill in the art will appreciate the wide variety of configurations that a carton/blister tray packaging system as described in the embodiments may take. The wide variety of configurations may enable the packaging of a diverse range of medical devices and combinations thereof.

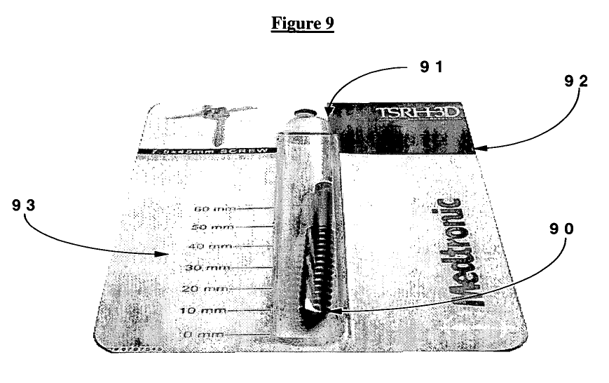

FIG. 8 illustrates a completely assembled carton/blister tray combination in accordance with the embodiments. A carton 80 is provided with at least one window 80a in the carton. Through the window, a blister tray with at least one cavity 81 is visible. The at least one cavity in the blister tray holds a medical device 82, in this case, a plate for spinal fixation procedures. As can be seen in FIG. 8, the one or more cavities in the blister tray are substantially visible through the one or more windows in the carton, so that the contents of the cavities may be capable of being inspected from outside of the package. The carton/blister tray packaging systems may enable the visual inspection of medical devices packaged therein without the necessity of removing the devices from the packaging. Thus, the carton/blister tray packaging systems may facilitate the visual inspection of medical devices packaged therein while maintaining the devices' sterility and authenticity (i.e., the packages' seals do not need to be broken in order to visually inspect the devices packaged therein).

The embodiments herein provide packaging systems for medical devices that may possess superior characteristics over previously known packaging systems. One advantageous quality of the packaging systems provided herein is that the medical device packaged therein may be at least substantially viewable from outside the package. This is desirable because visual inspection of the medical device may allow a surgeon or other medical practitioner to visually inspect and assess the suitability of the medical device for use in a given procedure without removing the device from its packaging. In this way, the authenticity and/or sterility of the medical device can be maintained even as the surgeon or other medical practitioner visually inspects the medical device. This may not only speed the selection of the medical device for use by the surgeon, but also reduce unnecessary re-packaging and re-sterilization of medical devices that are not found to be suitable for use in a given procedure or surgery after visual inspection of the device.

Additionally, the embodiments may provide improved methods to store and display the devices contained therein. Preferably, the cards and cartons as described in the embodiments may be hole-punched so that the cards and cartons may be hung on a peg. In this way, the hanging packaging systems may provide an improved method to package, store, and display medical devices.

Other embodiments provide packaging systems with measuring indicia on the outside of the card or carton and adjacent to the at least one window in the card or carton through which the cavity in the blister tray and the enclosed medical device may be observed. Preferably, the measuring indicia may be calibrated to one or more dimensions of the medical device or portion thereof contained in the package. The measuring indicia may be used to visually determine the size of the medical device by observing the measurements of its dimensions as indicated by the measuring indicia. The measuring indicia thus may enhance the utility of the packaging system in respect to a surgeon's ability to visually inspect the medical device and determine if it is appropriate for use in a medical procedure or surgery.

FIG. 9 illustrates an exemplary packaging system of the embodiments, wherein the packaging system comprises measuring indicia calibrated to a dimension of the medical device contained in the package. In FIG. 9, a medical device 90, in this case a bone screw, is packaged in a blister tray 91 that is secured to a card 92 having a window. Measuring indicia 93 are printed on the side of the card immediately adjacent to the blister tray. The measuring indicia are calibrated to the length of the bone screw from tip to shaft. Thus, a surgeon or other medical practitioner may easily ascertain the relevant dimension of the bone screw by comparing the medical device, which is viewable through the window, to the measuring indicia. Another dimension, not shown, which could be displayed on the card using measuring indicia is the bone screw's diameter. While FIG. 9 illustrates a card/blister tray packaging system, it will be appreciated that a carton/blister tray packaging system also is capable of being marked with measuring indicia that are calibrated to one or more dimensions of the medical devices packaged therein.

The foregoing detailed description is provided to describe the embodiments in detail, and is not intended to limit the embodiments. Those skilled in the art will appreciate that various modifications may be made to the embodiments without departing significantly from the spirit and scope thereof.

1. A packaging system for medical devices, comprising:
a blister tray that is at least semi-transparent and has at least one cavity, wherein the cavity is capable of containing the medical device, or a portion thereof; and
a card having at least one window;
wherein the at least one cavity in the blister tray protrudes through the at least one window in the card.

2. The packaging system of claim 1, additionally comprising a blister cover removably disposed over the blister tray and covering the at least one cavity in the blister tray.

3. The packaging system of claim 1, additionally comprising a backing card, wherein the blister tray is positioned between the backing card and the card having at least one window.

4. The packaging system of claim 1, wherein the card is folded into two sections, and the blister tray is positioned between the two sections of the folded card.

5. The packaging system of claim 1, wherein the card has measuring indicia on the card adjacent to the window.

6. The packaging system of claim 5, wherein the measuring indicia are calibrated to a dimension of the medical device or portion thereof.

7. The packaging system of claim 1, wherein the card comprises a paper product.

8. The packaging system of claim 2, wherein the blister cover comprises a foil.

9. The packaging system of claim 2, wherein the blister cover comprises a spun-bonded polymeric fabric.

10. The packaging system of claim 1, wherein the system is capable of maintaining a sterile environment in the at least one cavity of the blister tray.

11. A packaging system for medical devices, comprising:
a blister tray that is at least semi-transparent and has at least one cavity, wherein the cavity is capable of containing the medical device, or a portion thereof;
a blister tray closure removably disposed over the blister tray and covering the at least one cavity in the blister tray; and
a carton having at least one window;
wherein the blister tray is positioned inside of the carton, and the at least one cavity in the blister tray is substantially visible through the window in the carton.

12. The packaging system of claim 11, wherein the carton has measuring indicia on the outside of the carton and adjacent to the window.

13. The packaging system of claim 10, wherein the measuring indicia are calibrated to a dimension of a medical device.

14. The packaging system of claim 11, wherein the carton comprises a paper product.

15. The packaging system of claim 11, wherein the blister cover comprises a foil.

16. The packaging system of claim 11, wherein the blister cover comprises a spun-bonded polymeric fabric.

17. The packaging system of claim 11, wherein the system is capable of maintaining a sterile environment in the at least one cavity of the blister tray.
